(19) United States
(12) Patent Application Publication
(10) Pub. No.: US 2005/0075603 A1
(43) Pub. Date: Apr. 7, 2005

(54) Jet injector and method for its operation and production

(76) Inventor: Birger Hjertman, Vallingby (SE)

Correspondence Address: DINSMORE & SHOHL, LLP, 1900 CHEMED CENTER, 255 EAST FIFTH STREET, CINCINNATI, OH, 45202, US

(21) Appl. No.: 10/613,286

(22) Filed: Jul. 3, 2003

Related U.S. Application Data
(60) Provisional application No. 60/394,499, filed on Jul. 9, 2002.

(30) Foreign Application Priority Data
Jul. 3, 2002 (SE) 0202063-4

(51) Int. Cl. A61M 31/00

(52) U.S. Cl. 604/93.01; 604/146

(57) ABSTRACT

A jet injector comprising a housing (1) to be pressurized and holding a medical drug and which is defined by an enclosing periphery wall (3) and a bottom wall (4) having an internal surface (5) facing the interior of the housing (1) and opposite external surface (6). The bottom wall (4) has a through, truncated cone-shaped aperture (8) extending between said internal (5) and external (6) surfaces and through which said medical drug is expelled from the housing (1) and transformed into a thin jet stream (18) penetrating the skin of said person to be treated. An insert (9) having an essentially cone-shaped body (10) is arranged to be inserted into said housing (1) such that a portion (13) of said cone-shaped body (10), the periphery wall (39) of which is essentially congruent to the wall (12) of said aperture (8), is received in said aperture (8). At least one passage (7) is established between said portion (13) of said cone-shaped body (10) and the aperture wall (12) through which the medical drug is expelled from the housing (1). The drug then flows along said essentially cone-shaped body (10) to be delivered from the point (14) of the cone-shaped body (10) as a coherent, thin jet stream (18).

Present invention is directed to a jet injector for injecting a liquid medical drug through the skin of a person to be treated comprising a housing to be pressurized and holding said medical drug and which is defined by an enclosing periphery wall and a bottom wall having an internal surface facing the interior of the housing and an opposite external surface, the bottom wall has at least one through passage extending between said internal and external surfaces and through which, when the injector is used, said medical drug is expelled from the housing and transformed into a thin jet stream penetrating the skin of said person. The invention is also directed to a method for operating such an injector.

This invention is also directed to a method for producing a jet injector for injecting a liquid medical drug through the skin of a person to be treated comprising a housing to be pressurized and holding said medical drug and which is defined by an enclosing periphery wall and a bottom wall having at least one through passage.

Existing needleless jet injectors for injection of a liquid medical drug creates a jet stream penetrating the skin of a person to be treated by the inherent force of said stream. Those injectors generally include a high pressure housing having one or more openings for an exit jet stream. Said opening or openings are quite narrow, typically in the order of some tenth of a millimetre. Such openings are very difficult to produce with close tolerances, both rationally and reproduceably, eg by injection moulding of plastic material. Besides, it is difficult to produce openings having such a design and smoothness that the exit jet stream will be coherent and accurately confined to achieve best possible skin penetration effect.

PCT application WO 01/05454 is directed to a needleless syringe comprising an injector with nested elements, the inner elements of which having a number of small chanels formed on their periphery surfaces for conducting a medical drug from a pressurized chamber, through said number of chanals and to the skin of a patient so that a plurality of drug streams penetrate the skin. The mouth of each chanal is in contact with the skin and the drug streams are injected directly into the skin, ie without any gaps therebetween. The fineness of said drug streams are determined by the size of the chanals and the section area of the chanals has a minimal dimension. If the dimension is too small the flow resistance in the chanals will be too high and reduces the flow rate of the drug jeopardizing the desired penetration effect.

It is an object of present invention to provide a jet injector having the capacity to create a jet stream with adjustable fineness and at the same time accurate flow rate to achieve a desired penetration effect.

It is an other object of present invention to provide a method for inexpensive, reliable and easy manufacturing of a jet injector.

A further object of present invention is to provide a jet injector having an outlet which not will be contaminated by the skin of a person during injection.

These and other objects of present invention have been fulfilled by a jet injector according to the first paragraph of this description, characterized in that said through passage includes a flow confining restriction to develop a high pressure in the medical drug expelled from the housing, in that a body is connected to said passage, said body is tapering in a direction away from the passage and terminates in a point, and in that said body has a periphery surface receiving the expelled medical drug and guiding it towards said point to create a coherent jet stream emerging from said point.

A method for producing a jet injector according to present invention and stated in the second paragraph of the preamble is characterized by by the steps:
(i) providing a mould for injection moulding of said periphery wall and bottom wall and a through aperture in said bottom wall;
(ii) machining portions of the wall of the mould forming an interior surface of said bottom wall and/or said aperture of the bottom wall such that recesses are created in the wall of the mould, eg by milling, cutting, engraving, spark machining or etching;
(iii) injection moulding, whereby protrusious are formed on said interior surface and/or on a wall of said aperture;
(iv) providing a mould for injection moulding of an insert having an essentially cone-shaped body intended to be received in said aperture and having a periphery surface essentially congruent to said wall of the aperture;
(v) in addition to or instead of step (ii) machining portions of the wall of the mould forming said periphery surface such that recesses are created in the wall of the mould, eg by milling, cutting, engraving, spark machining or etching;
(vi) injection moulding, whereby protrusious are formed on said periphery surface; and
(vii) positioning said essentially cone-shaped body into said aperture by inserting said insert into said housing, its point first, so that said protrusious contact an opposite surface or wall, thereby creating a passage designed as a substantially ringshaped gap between said aperture and said body.

An alternative method for manufacturing a jet injector according to the invention and the second paragraph is characeterized by following steps:
(i) providing a mould for injection moudling of said periphery wall and bottom wall and a through aperture in said bottom wall;
(ii) injection moulding,
(iii) machining a wall of said formed aperture such that grooves are created in said wall, eg by milling, cutting, engraving or etching, said grooves extending from an internal surface of said bottom wall to its external surface;
(iv) providing a mould for injection moulding of an insert having an essentially cone-shaped body intended to be received in said aperture and having a periphery surface essentially congruent to said wall of the aperture;
(v) in addition to or instead of step (iii) machining said periphery surface such that grooves extending from an internal surface of said bottom wall to its external surface when said insert is positioned into said aperture according to step (vi);
(vi) positioning said essentially cone-shaped body into said aperture by inserting said insert into said housing, its point first, so that said periphery surface of the cone-shaped body contacts said wall of the aperture creating a passage designed as a number of tube-like flutes between said aperture and said body.

Further developments of present invention are stated in the subclaims.

Preferred embodiments of the invention are illustrated below with reference to the accompanying drawings, in which:

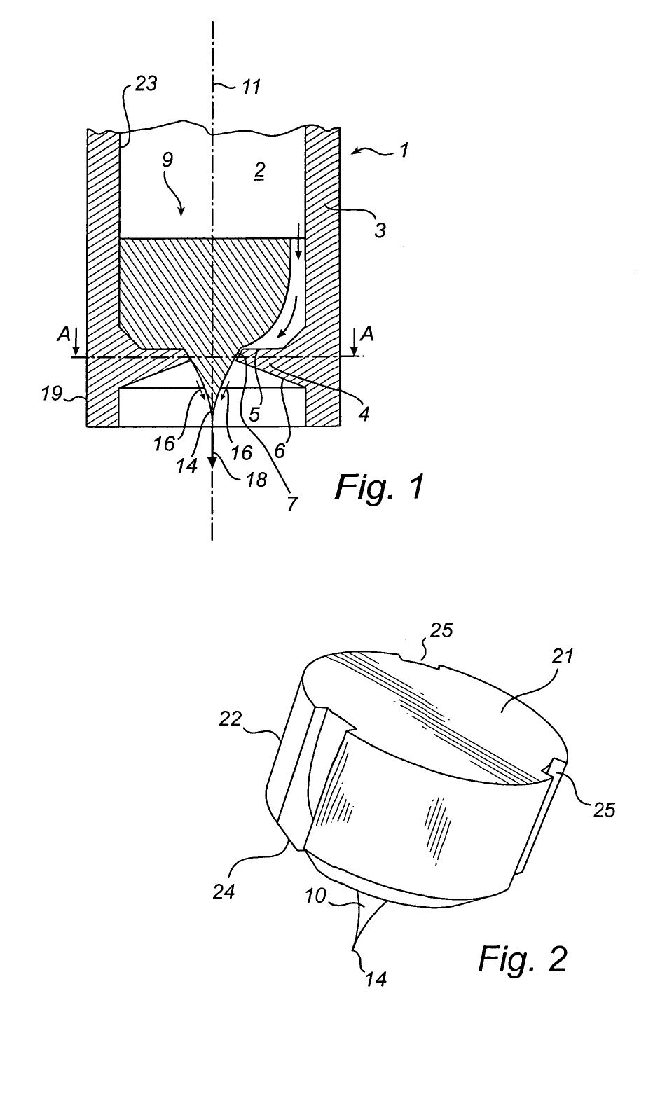

FIG. 1 is a fragmentary, longitudinal section view of a jet injector according to one embodiment of present invention;

FIG. 2 is a perspective view of an insert arranged to be inserted in a housing of jet injector according to FIG. 1;

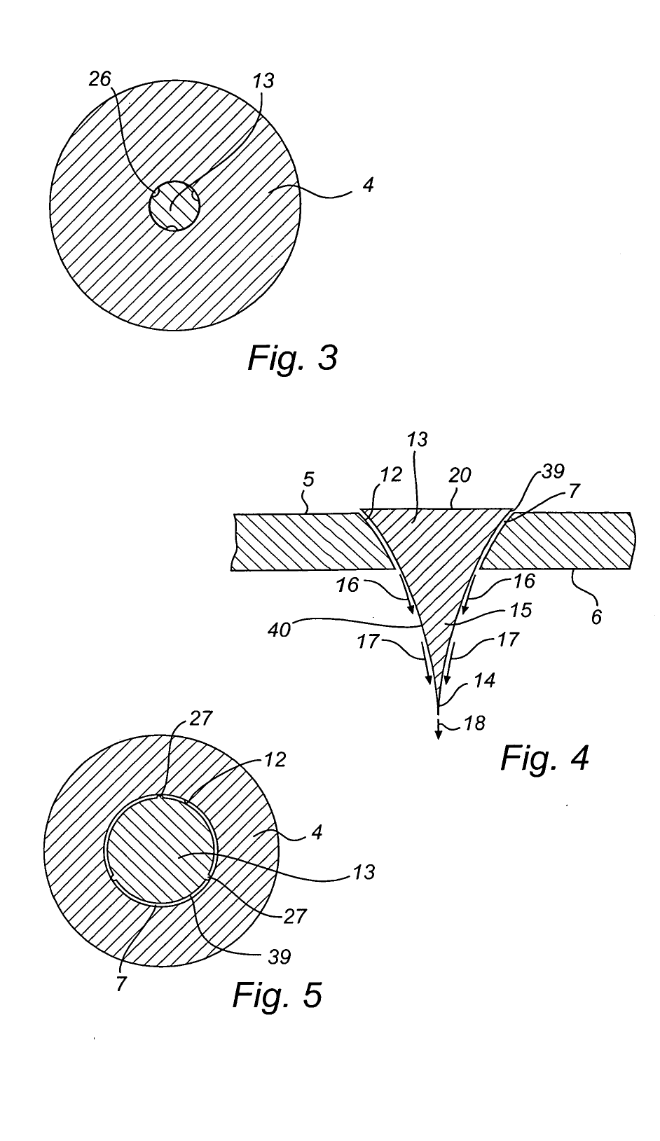

FIG. 3 is a cross section view along line A-A;

FIG. 4 is an enlarged view of a portion of the insert and a surrounding wall portion according to the embodiment of FIG. 1 and 2 illustrating the flow of an expelled medical drug, whereby the gap between these portions is exaggerated for clarity reasons;

FIG. 5 is a view similar to that of FIG. 3 and showing a second embodiment of the invention;

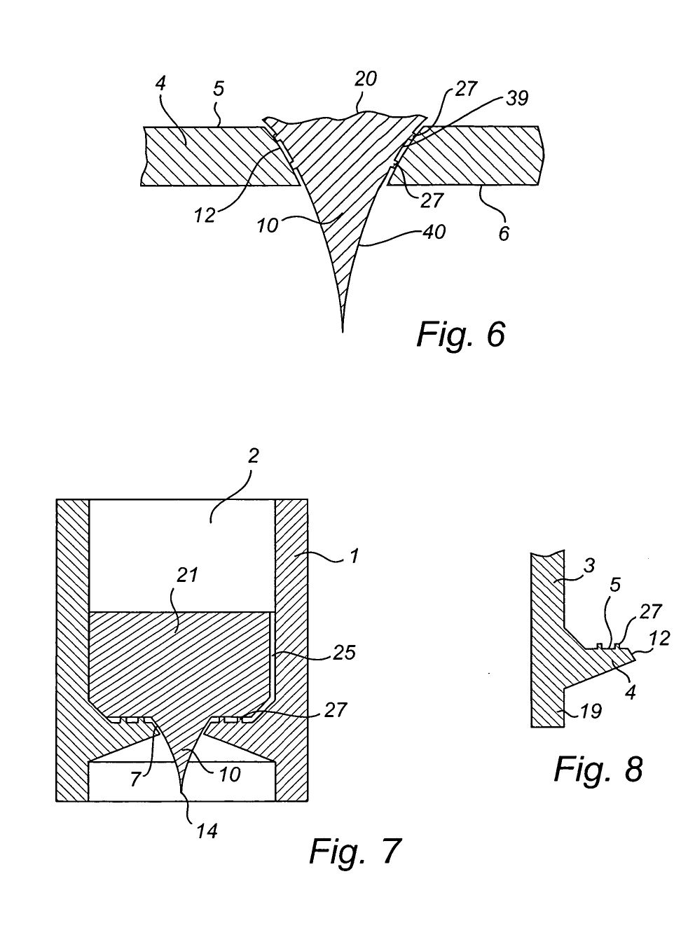

FIG. 6 is a fragmentary, longitudinal section view of the embodiment in FIG. 5;

FIG. 7 is a view similar to that of FIG. 1 showing a third embodiment of the invetion;

FIG. 8 is an enlarged detail view of a part of the wall portion according to the embodiment in FIG. 7;

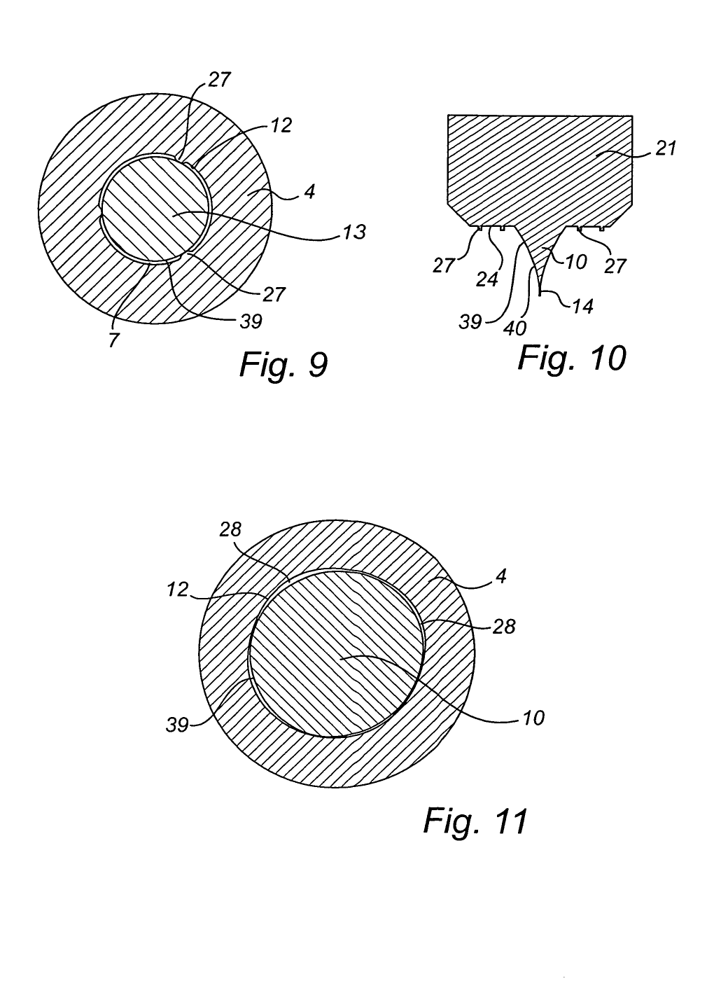

FIG. 9 is a view similar to that of FIG. 3 and showing a fourth embodiment of present invention;

FIG. 10 is an enlarged detail view of a part of the insert according to a fifth embodiment of the invention;

FIG. 11 is a view similar to that of FIG. 3 schematically depicting a sixth emobodiment of present invention;

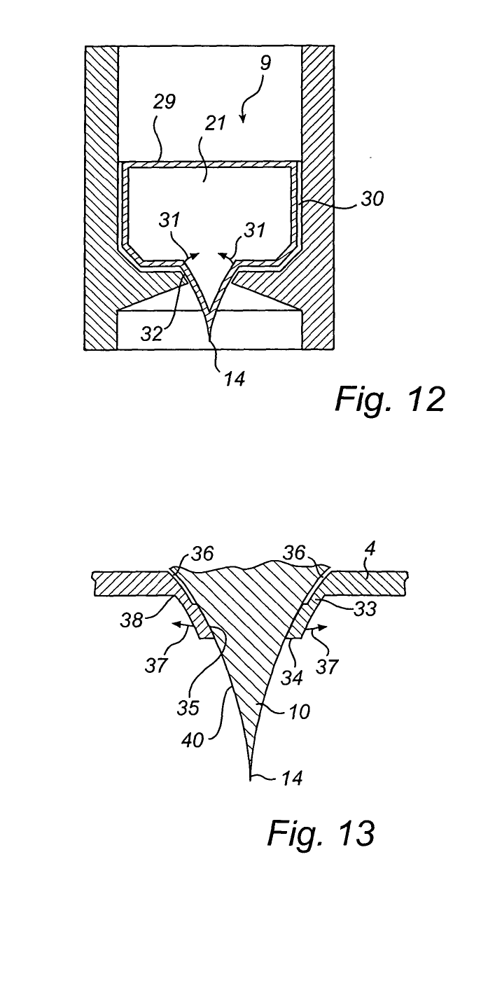

FIG. 12 in a view similar to that of FIG. 1 illustrates a seventh embodiment of the invention; and

FIG. 13 in a view similar to that of FIG. 2 illustrates an eighth embodiment of the invention.

Present invention concerns a jet injector for delivering a liquid medical drug or preparation to a person to be treated by creating or forming a jet stream of the liquid drug, which jet stream has an accurate fineness and force to penetrate the skin of said person and enter his body.

Referring first to FIG. 1-3 an jet injector according to present invention generally comprises a housing or reservoir 1 holding a medical drug 2 to be dispensed, when pressurized in an arbitrary way known by a man skilled in the art, eg by means of a piston device (not shown). The housing 1 is defined by an enclosing periphery wall 3, an unillustrated top portion (not part of the invention) and a bottom wall 4. The bottom wall 4 has an internal surface 5 facing the interior of the housing 1 and an opposite external surface 6. A through passage 7 (cf FIG. 4) is provided in the bottom wall 4 extending between said internal and external surfaces 5 and 6, resp., and when using the injector said medical drug 2 is expelled from the housing 1. Preferably, the through passage 7 is a gap between a through hole or aperture 8 (cf FIG. 5) and an insert 9 placed inside the aperture 8. In the illustrated embodiment there is a single aperture centrally positioned on a symmetry axis 11 of said housing 1 and the aperture 8 has a truncated cone-shaped configuration converging in a direction away from the housing 1 and is defined by a wall 12. The insert 9 has an essentially cone-shaped body 10 and is arranged to be inserted into the housing such that a portion 13 of said essentially cone-shaped body 10, the periphery surface 39 of which is generally congruent to the aperture wall 12, is received in the aperture 8. Said body 10 tapers in a direction away from the passage 7 and terminates in a sharp point or tip 14 at a distance from the bottom wall 4 outside the housing and positioned on said symmetry axis 11. It is preferred that the tip is sharp although it is also possible to have it slightly truncated for edge release of the liquid. Since both the through passage 7 and the body 10 have substantially cone-shaped outer surfaces the width of the passage can easily be adjused by relative axial displacement or positioning between these parts.

Said through passage or gap 7 between the aperture wall 12 and said portion 13 of the essentially cone-shaped body 10 serves as a flow confining restriction to develop a high pressure in the medical drug expelled from said housing 1, when using the jet injector. The remaining portion 15 of the essentially cone-shaped body, ie the portion between its portion 13 opposite the aperture wall 12 and its point 14, has a periphery surface 40 receiving or catching the medical drug flowing from the passage 7 and guiding it towards said point 14 to create or form a coherent, thin jet stream emerging from the point 14. At least an area including the point 14 of said body 10 has a concave periphery surface, or concave periphery line when seen in cross-section through the axis, and preferably all of the periphery surface of said body portion 15 between the external surface 6 of the bottom wall 4 and the point 14 is concave, cf FIG. 4. This figure illustrates schematically the flow of said expelled drug. A stream or substreams 16 of a medical drug pressurized in said passage 7 flow(-s) along the periphery surface 40 of said portion 15 attracted to said surface by the so called Coanda effect. When approaching said point 14 the substreams coalesence or are focused before reaching the point 14 creating a substantially homogeneous flow 17 arriving to the point 14 and forming a coherent, thin jet stream 18 emerging from said point 14. The fineness of said jet stream is related to said flow confining restriction, ie the pressure of the stream(-s) 16. The curvature of the concave surface or line can vary somewhat. In order to have a coherent stream it is preferred that the surface is designed so as to give a decreasing angle between the converging streams towards the tip and preferably an almost parallel or substantially parallel flow at the very tip, e.g. by letting the tangential lines of the surface close to the tip substantially coincide. The converging concave lines of the cross-section can for exmple be be segments of a circle but it is preferred that the curvature changes towards to tip, which can be obtained if instead the lines are shaped as segments of an oval, parabolic or hyperbolic curve or intermediates therbetween. Preferably the segments are mirror images with respect to the axis 11, preferably also arranged to give a decreasing curvature towards the tip and most preferably placed so as to give the abovesaid substantially parallel lines at the tip.

Referring again to FIG. 1 a positioning element 19 protrudes from said bottom wall 4 and preferably but not mandatory terminates at a level beyond or at said point 14 of the essentially cone-shaped body 10. A position beyond the tip may serve to keep a distance between the tip and the target surface, e.g. to let the stream form before hitting the target, whereas a position at or behind the tip may serve to allow the tip to contact the target surface, e.g. to act as a short needle for severing the target tissue and reduce the penetrating requirements on the liquid jet. The positioning element 19 is intended to be placed on the skin of the person to be treated when injecting the medical drug. As depicted in this figure the periphery wall 3, the bottom wall 4 and the positioning element 19 of the jet injector are made in one piece of resin, preferably polycarbonate resin, ie by injection moulding. The relatively large aperture 8 in the bottom wall 4 is rather easy to manufacture with accurate tolerances, either during said moulding or afterwards by machining, compared to the small borings in the art for transferring a medical drug.

As discussed above the insert 9 comprises an essentially cone-shaped body 10, the base 20 of which is positioned substantially at the level of said internal surface 5 of the bottom wall 4, when the injector is assembled, cf FIG. 4. However, and preferably, the insert 9 also comprises a head body 21, cf FIGS. 1 and 2, arranged to be inserted in said housing 1 close to said bottom wall 4 and connected to said essentially cone-shaped body 10. Preferably the head body 21 and the essentially cone-shaped body 10 are made in one piece of resin, suitably polycarbonate plastic, ie by injection moulding. The head body 21 has a periphery surface 22 matching the inner surface 23 of said periphery wall 3, a bottom surface 24 having a configuration adapted to said internal surface 5 of the bottom wall 4 and a number of flutes 25 formed in the head body 21 and connecting the interior of the housing 1 with said aperture 8, directly or indirectly as will be discussed below, said flutes 25 extending along the peripheral surface 22, the bottom surface 24 and terminating at said internal surface 5 inside the aperture area.

Referring especially to FIG. 3 said through passage 7 is a number of grooves 26 formed in said upper portion 13 of the essentially cone-shaped body 10 extending between the lower portion 15 of the body 10 and the ends of said flutes 25, thereby communicating the interior of the housing 1 with said portion 15 and ultimately with the jet stream forming point 14. Alternately or in addition a number of grooves are formed in the aperture wall 12 extending from its internal surface 5 to its external surface 6, not shown. In this last mentioned case a key means (not shown but known to a man skilled in the art) is arranged to position the outlets of the flutes 25 in register with the inlets of the grooves 26. Each flute 25 has a section area larger than that of a groove 26, said flutes 25 passing pressurized medical drug from the interior of the housing 1 into relevant ones of said grooves 26.

In another embodiment of present invention said passage 7 is configurated substantially like a ring-shaped gap instead of a plurality of grooves. According to this embodiment a number of spacing means are provided between said insert 9 and said bottom wall 4 and/or between said insert 9 and said aperture wall 12, whereby a ring-shaped gap is formed between the periphery surface 39 of said essentially cone-shaped body and the aperture wall 12.

Referring first to FIGS. 5 and 6 showing a cross section view of the aperture perpendicular to said symmetry axis and a section view along said symmetry axis 11, resp., said spacing means are protrusions 27 projecting from the peripheral surface 39 of the upper portion 13 of said essentially cone-shaped body 10 engaging the wall 12 of said aperture 8. The protrusions 27 are at least three in number extending substantially from the internal surface 5 to the external surface 6 of the wall 4, when the insert 9 is mounted. When pin-shaped the protrusions 27 are preferably arranged in pairs, cf FIG. 6. As an alternative or in addition the protrusions are provided on and projecting from the wall 12 of said aperture 8 engaging said peripheral surface 39 of the upper portion 13 (cf FIG. 9). The protrusions 27 are bosses, pins, studs, ribs, ridges or the like integrated in the surface/wall from which they project. FIG. 7 depicts spacing means as protrusions provided between the bottom surface 24 of said head body 21 and the internal surface 5 of said bottom wall 4. In this embodiment also said restriction is achieved as a ring-shaped gap between the aperture wall 12 and said periphery surface 39 of the essentially cone-shaped body 10. FIG. 8 illustrates, in an enlarged scale, protrusions 27 projecting from said internal surface 5 of the bottom wall 4 and FIG. 9 illustrates, in a similar scale, protrusions 27 projecting from the bottom surface 24 of said head body 21. FIG. 10 depicts an alternative where protrusions 27 are integral with the head body 21 of the insert 9. The protrusions may otherwise be similar those mentioned in connection with FIG. 8 and 9.

FIG. 11 depicts schematically still another embodiment of present invention. Said cone-shaped body 10, and specially its upper portion 13, is manufactured with coarse tolerances and so is the aperture 8 creating gaps occuring between the periphery surface 39 of the upper portion 13 of said essentially cone-shaped body 10 and the surrounding wall 12 of said aperture 8. These gaps 28 cooperate to constitute a passage 7 for transferring medical drug from said housing 1 to said jet stream forming portion 15 of the insert 9.

Referring now to FIGS. 12 and 13 embodiments equipped with flexible walls are shown constituting a flow confining restriction passage, the magnitude of the restriction is related to the pressure in said housing 1.

In the above illustrated and discussed embodiments the insert 9 is a solid and homogeneous body. However, and according to the embodiment of FIG. 12, said insert 9 is a hollow body confined by a pliable, elastic thin-walled shell 29. The head body 21 of the insert 9 has a diameter smaller than the inner diameter of the housing 1 forming a ring-shaped chanal 30 therebetween. Preferably the bottom wall 24 of the head body 21 forms an angle with said symmetry axis 11 more acute than the angle between the internal surface 5 of said bottom wall 4 and the symmetry axis to establish a flow path from the housing 1 to the aperture 8. When the medical drug in the housing is pressurized it flows into the space between the head body 21 and the bottom wall 4 causing the wall of said essentially cone-shaped body 10 to deflect inwardly, in the direction of the arrows 31, said point 14 area serving as a pivot, such that a gap 32 is temporarily formed between the cone-shaped body 10 and said aperture wall 12 admitting a medical drug flow therebetween.

FIG. 13 shows a further embodiment of present invention working with flexible walls. The essential difference between this embodiment and the previous ones is that a central port of the bottom wall 4 is configurated like a truncated funnel or hopper instead of plate having a converging aperture. A pliable elastic wall 33 formed as a truncated funnel and converging outwardly from said housing 1 is depending from the bottom wall 4 and is terminated in a bottom surface 34 forming a sharp edge in contact with the essentially cone-shaped body 10. The elastic wall 33 is integrated with the bottom wall 4 and in its upper portion a number of blind grooves or recesses 36 are formed. As in the above discussed embodiments, except the one in FIG. 12, a solid insert 9 with or without a head body 21 is to be positioned inside said wall 33. When pressurized, the medical drug in the housing 1 will flow into said grooves 36 causing the flexible wall 33 to deflect outwardly, in the direction of the arrows 37, the junction between the bottom wall 4 and said flexible wall 33 serving as a pivot. Thus, the lower portion of said wall 33 will be separated from the essentially cone-shaped body 10 temporarily forming a passage 7 therebetween letting the medical drug through.

Present invention is also directed to methods for producing a jet injector discussed above (excluding the embodiments of FIGS. 11 and 12).

A method for producing, for example, a jet injector illustrated in FIGS. 5-10 includes following steps.
(i) Manufacture a mould (not shown) for injection moulding of said periphery wall 3 and bottom wall 4 and a through aperture 8 in said bottom wall. To manufacture a mould to produce a specified object is common knowledge for a man skilled in the art and is therefore not discussed further herein.
(ii) Machining portions of the wall of the mould forming an internal surface 5 of said bottom wall 4 and/or said aperture 8 of the bottom wall such that recesses are created in the wall of the mould, eg by milling, cutting, engraving, spark machining or etching. By machining it is possible to create very small (and larger) recesses having close tolerances.
(iii) Injection mould to form protrusions 27 on said internal surface 5 and/on the wall 12 of the aperture 8.
(iv) Manufacture a mould (not shown) for injection moulding of an insert 9 having an essentially cone-shaped body 10 intended to be received in said aperture 8 and having a periphery surface 39 essentially congruent to said wall 12 of the aperture; cf step (i).
(v) In addition to or instead of step (ii) machin portions of the wall of the mould forming said periphery surface 39 such that recesses are created in the wall of the mould, eg by milling, cutting, engraving, spark machining or etching.
(vi) Injection mould to form protrusions 27 on said periphery surface 39.
(vii) Remove the accordingly moulded housing 1 (and integrated positioning element 19) and insert 9 from their moulds and place said essentially cone-shaped body 10 into said aperture 8 by inserting said insert 9 into the housing, its point 14 first, so that said protrusious 27 contact an opposite surface or wall (5,12,24,39), thereby creating a passage 7 designed as a substantially ringshaped gap between said aperture 8 and said body 10.

Further, a method for producing a jet injector according to FIGS. 1-3 comprises following steps.
(i) Manufacture a mould as in previous step (i).
(ii) Injection mould.
(iii) Machin the wall 12 of said formed aperture 8 such that grooves 26 are created in said wall, eg by milling, cutting, engraving or etching, said grooves extending from an internal surface of said bottom wall to its external surface.
(iv) Manufacture a mould as in step (iv) of previous method.
(v) In addition to or instead of step (iii) machin said periphery surface 39 such that grooves 26 extend from the internal surface 5 of said bottom wall 4 to its external surface 6, when said insert 9 is positioned into said aperture 8 according to step (vi).
(vi) Position the essentially cone-shaped body 10 into said aperture 8 by inserting said insert 9 into said housing 1, its point 14 first, so that said periphery surface 39 of the cone-shaped body 10 contacts said wall 12 of the aperture 8 creating a passage. 7 designed as a number of tubelike flow pathes between said aperture and said body. Each of said flow pathes is thus confined of a groove 26 and an opposite surface area of the aperture wall 12 or said periphery surface 39.

As will be recognized by a man skilled in the art the embodiment according to FIG. 13 is produced in a way similar to the last mentioned method regarding the grooves 36 (step v).

The insert 9 of the jet injector in FIG. 12 is preferably produced by blow moulding. A related gas inlet (not shown) is provided in the upper (horisontal) wall of the shell 29. After having removed the insert from the mould said inlet is airtight sealed in a per se known appropriate way.

1-25. (canceled)

26. Jet injector for injecting a liquid medical drug through the skin of a person to be treated, comprising a housing adapted to be pressurized and hold said medical drug, defined by an enclosing periphery wall and a bottom wall having an internal surface facing the interior of the housing and an opposite external surface, the bottom wall having at least one through passage extending between said internal and external surfaces and through which, when the injector is used, said medical drug is adapted to be expelled from the housing and transformed into a thin jet stream penetrating the skin of said person, wherein said through passage includes a flow confining restriction to develop a high pressure in the medical drug expelled from the housing, wherein a body is connected to said passage and tapers in a direction away from the passage and terminates in a point, and wherein said body has a periphery surface adapted to receive the expelled medical drug and guide the expelled medical drug towards said point to create a coherent jet stream emerging from said point.

27. Jet injector according to claim 26, wherein said bottom wall has a through, truncated cone-shaped aperture defined by a wall, wherein an insert having an essentially cone-shaped body is arranged to be inserted into said housing such that a portion of said cone-shaped body, the periphery wall of which is essentially congruent to said aperture wall, is received in said aperture and wherein at least one passage is established between said portion of said cone-shaped body and the aperture wall through which the medical drug is adapted to be expelled from the housing, whereby said drug then flows along said essentially cone-shaped body to be delivered from said point of the cone-shaped body as a coherent, thin jet stream.

28. Jet injector according to claim 27, wherein at least a part of said point of said essentially cone-shaped body has a concave periphery.

29. Jet injector according to claim 27, wherein said periphery surface of said essentially cone-shaped body, from its point to said passage, is concave.

30. Jet injector according to claim 27, wherein said essentially cone-shaped body is terminated in a sharp point.

31. Jet injector according to claim 27, wherein a positioning element protrudes from said bottom wall and terminates at a level beyond or at said point of the essentially cone-shaped body, and said positioning element is adapted to be placed on the skin of the person to be treated when injecting the medical drug.

32. Jet injector according to claim 31, wherein said periphery wall, bottom wall and positioning element of the jet injector are made in one piece of resin.

33. Jet injector according to claim 32, wherein the resin comprises polycarbonate.

34. Jet injector according to claim 27, wherein said insert is said essentially cone-shaped body, the base of which is positioned at the level of said internal surface of the bottom wall, when the injector is assembled.

35. Jet injector according to claim 27, wherein said insert comprises a head body arranged to be inserted in said housing close to said bottom wall and connected to said essentially cone-shaped body.

36. Jet injector according to claim 35, wherein said head body and the essentially cone-shaped body are made in one piece of resin.

37. Jet injector according to claim 36, wherein the resin comprises polycarbonate.

38. Jet injector according to claim 27, wherein said bottom wall is perforated by a single aperture centrally positioned on a symmetry axis of said housing, said point of the essentially cone-shaped body being positioned on said symmetry axis when assembled.

39. Jet injector according to claim 27, wherein a number of spacing means are provided between said insert and said bottom wall and/or between said insert and said aperture wall, whereby a ring-shaped gap is formed between the periphery surface of said essentially cone-shaped body and the aperture wall.

40. Jet injector according to claim 39, wherein said spacing means are positioned between said internal surface of the bottom wall and an opposite surface of said head body.

41. Jet injector according to claim 40, wherein said spacing means comprise protrusions projecting from said internal surface of the bottom wall and/or from said surface of the head body.

42. Jet injector according to claim 41, wherein said spacing means are bosses, pins, studs, ribs, or ridges, integrated in the surface or wall from which they project.

43. Jet injector according to claim 39, wherein said spacing means are positioned between said periphery surface of the essentially cone-shaped body and said aperture wall.

44. Jet injector according to claim 43, wherein said spacing means comprise protrusions projecting from said periphery surface and/or said aperture wall.

45. Jet injector according to claim 44, wherein said spacing means are bosses, pins, studs, ribs, or ridges, integrated in the surface or wall from which they project.

46. Jet injector according to claim 27, wherein said passage comprises a number of grooves formed in said aperture wall and/or in said periphery surface of the essentially cone-shaped body.

47. Jet injector according to claim 46, wherein a number of flutes are formed in a surface of said head body facing said periphery wall of the housing and in its surface facing said bottom wall, said flutes having a section area larger than that of said grooves, said flutes being adapted to pass pressurized medical drug from the interior of said housing into relevant ones of said grooves.

48. Jet injector according to claim 27, wherein said insert is a hollow body confined by a pliable, elastic thin-walled shell, said shell being adapted to deflect when subjected to pressurized medical drug such that a gap is temporarily formed between the cone-shaped body and said aperture wall admitting a medical drug flow therebetween.

49. Jet injector according to claim 27, wherein a pliable, elastic wall formed as a truncated funnel depends from said bottom wall and constitutes said aperture, the upper part of said pliable wall having a number of blind grooves and being adapted to deflect when subjected to pressurized medical drug such that a gap is temporarily formed between the cone-shaped body and said pliable wall admitting a medical drug flow therebetween.

50. Jet injector according to claim 27, wherein said essentially cone-shaped body is solid and said body and said aperture are manufactured with coarse tolerances, said passage being formed by gaps occurring between said periphery surface of the body and said aperture wall.

51. A method for producing a jet injector for injecting a liquid medical drug through the skin of a person to be treated, the jet injector comprising a housing adapted to be pressurized and hold said medical drug, defined by an enclosing periphery wall and a bottom wall having at least one through passage, the method comprising:
(i) providing a mould for injection moulding of said periphery wall and bottom wall and a through aperture in said bottom wall;
(ii) machining portions of the wall of the mould forming an internal surface of said bottom wall and/or said aperture of the bottom wall such that recesses are created in the wall of the mould;
(iii) injection moulding, whereby protrusions are formed on said internal surface and/or on a wall of said aperture;
(iv) providing a mould for injection moulding of an insert having an essentially cone-shaped body intended to be received in said aperture and having a periphery surface essentially congruent to said wall of the aperture;
(v) in addition to or in place of step (ii), machining portions of the wall of the mould forming said periphery surface such that recesses are created in the wall of the mould;
(vi) injection moulding, wherein protrusions are formed on said periphery surface; and
(vii) positioning said essentially cone-shaped body into said aperture by inserting said insert into said housing, a point thereof first, so that said protrusions contact an opposite surface or wall, thereby creating a passage designed as a substantially ring-shaped gap between said aperture wall and said periphery surface of the essentially cone-shaped body.

52. Method according to claim 51, wherein the recesses are created by milling, cutting, engraving, spark machining, or etching.

53. A method for producing a jet injector for injecting a liquid medical drug through the skin of a person to be treated, the jet injector comprising a housing adapted to be pressurized and hold said medical drug, defined by an enclosing periphery wall and a bottom wall having at least one through passage, the method comprising:
(i) providing a mould for injection moulding of said periphery wall and bottom wall and a through aperture in said bottom wall;
(ii) injection moulding;
(iii) machining a wall of said formed aperture such that grooves are created in said wall, said grooves extending from an internal surface of said bottom wall to its external surface;
(iv) providing a mould for injection moulding of an insert having an essentially cone-shaped body intended to be received in said aperture and having a periphery surface essentially congruent to said wall of the aperture;
(v) in addition to or in place of step (iii), machining said periphery surface such that grooves extend from an internal surface of said bottom wall to its external surface, when said insert is positioned into said aperture according to step (vi);
(vi) positioning said essentially cone-shaped body into said aperture by inserting said insert into said housing, a point thereof first, so that said periphery surface of the cone-shaped body contacts said wall of the aperture creating a passage designed as a number of tube-like flow paths between said aperture and said body. 54. (NEW) Method according to claim 52, wherein the grooves are created by milling, cutting, engraving, spark machining, or etching. 55. (NEW) A method for forming a thin jet stream of a liquid medical drug for injection into a target by means of a jet injector comprising a housing adapted to be pressurized and holding said medical drug and defined by an enclosing periphery wall and a bottom wall having an internal surface facing the interior of the housing and an opposite external surface, the bottom wall having at least one through passage extending between said internal and external surfaces and through which, when the jet injector is used, said medical drug is expelled from the housing, the method comprising:
(i) pressurizing said housing to expel said medical drug from the housing and through said passage;
(ii) restricting the flow of said medical drug from said housing to develop a high pressure in the medical drug flowing from said passage;
(iii) guiding the outflowing medical drug to an essentially cone-shaped body provided on said jet injector and connected to said passage, said body tapering in a direction away from the passage and terminating in a point;
(iv) causing the expelled medical drug to flow along a periphery surface of said body towards the point;
(v) focusing, at said point, the expelled medical drug into a homogeneous flow; and
(vi) forming a coherent jet stream emerging from said point.
